(19) United States
(12) Patent Application Publication
(10) Pub. No.: US 2025/0360488 A1
(43) Pub. Date: Nov. 27, 2025

(54) BIO-BASED AND BIO-DEGRADABLE SUPERABSORBENT POLYMER AND METHODS FOR PREPARING THE SAME

(71) Applicant: Nano and Advanced Materials Institute Limited, Hong Kong (HK)

(72) Inventors: Mimi HETTI, Hong Kong (HK); Ching Man CHAN, Hong Kong (HK); Jiawen HUANG, Hong Kong (HK); Wei LI, Hong Kong (HK); Yeuk Tin LAU, Hong Kong (HK)

(21) Appl. No.: 19/218,676

(22) Filed: May 27, 2025

Related U.S. Application Data
(60) Provisional application No. 63/652,111, filed on May 27, 2024.

(51) Int. Cl. B01J 20/24; B01J 20/28; B01J 20/30; B01J 20/32

(52) U.S. Cl. CPC B01J 20/24; B01J 20/28016; B01J 20/2803; B01J 20/3021; B01J 20/3042; B01J 20/3085; B01J 20/3212; B01J 20/3219; B01J 20/3293

(57) ABSTRACT

The present invention relates to a bio-based and biodegradable superabsorbent polymer (SAP) comprising sodium alginate, modified cellulose, at least one activation agent, and at least one crosslinking agent. The SAP exhibits a high bio-based content of up to 93% and fulfills industrial composting standards such as EN 13432. The crosslinking reactions are performed under mild conditions, including ambient temperatures and neutral to alkaline pH, eliminating the need for high thermal energy. The resulting SAP granules demonstrate excellent liquid absorption performance, rapid swelling, high retention, and no observed toxicity or skin sensitization. The invention provides a sustainable alternative to petroleum-based SAPs for use in hygiene products, agriculture, and other disposable applications.

### CROSS-REFERENCE TO RELATED APPLICATIONS

The present application claims the priorities from the U.S. provisional patent application Ser. No. 63/652,111 filed May 27, 2024, and the disclosure of which is incorporated herein by reference in its entirety.

### FIELD OF THE INVENTION

The present invention relates to a formulation of a bio-based and bio-degradable superabsorbent polymer and a method of preparing the same, and particularly, but not exclusively, to a formulation of a bio-based and bio-degradable superabsorbent polymer with raw materials from bio-sources.

### BACKGROUND OF THE INVENTION

A superabsorbent polymer (SAP) is a water-absorbing hydrophilic and crosslinked polymer that can absorb and retain enormous amounts of a liquid relative to its own mass without dissolving. Over 90% of the SAP is applied to disposables, for example, adult incontinence products, baby diapers, female hygiene products, and absorbent pads. Polyacrylate (PAA), which is from non-renewable and non-degradable petroleum-based acrylic acid (AA), is almost the only SAP material in the market. Due to the global eco-friendly incentive of reduction of reliance on petroleum and elimination of persistent waste, the market need for bio-based and bio-degradable SAP material from renewable sources and as alternatives to petroleum-based PAA and which is becoming increasingly popular.

For example, U.S. patent application numbers U.S. Pat. Nos. 5,736,595A and 5,721,295A disclosed a 2-components SAP which contained carboxymethyl cellulose and water swellable polymers but without verified bio-degradation property and low overall bio-based content. U.S. patent application number U.S. Pat. No. 7,144,957B2 used purely bio-based starch as the backbone to graft monomers onto the backbone by graft-copolymerization but the monomers are also mainly petroleum based and the resultant SAP also could not show bio-degradability.

Among those inventions of both bio-based and bio-degradable SAPs, CN117545797A, KR20230009824A and US20230087087A1 disclosed the method of making starch and chitosan-based SAP which could achieve over 90% degradation within 6 months according to ISO 14855. This invention used carboxylic anhydride as the first crosslinking agent and epoxy-based compound as the second surface crosslinking agent, which requires high reaction temperature of 80-140° C.

In addition, US20200054782A1 disclosed a starch-based SAP without synthetic polymer and that SAP contained 60-95% bio-based carbon and could achieved over 60% bio-degradation in 90 days according to ASTM D5388. The crosslinking agents used in invention, such as citric acid, succinic acid, magnesium acetate, aluminum lactate, aluminum hydroxide, zinc chloride, and glycerol, also required a high crosslinking temperature of over 85° C. Other crosslinking or surface crosslinking agents, such as maleic anhydride (mentioned in JP4880144B2), alkylene carbonates, various oxazolidinones (mentioned in US20180043052A1), boric acid, epichlorohydrin, divinyl sulfone (mentioned in JP4685017B2), acrylate ester of a polyalcohol, ethoxylate glycerin (mentioned in EP2714775A1 and WO2012164018A1), and guanidinated polysaccharides (mentioned in U.S. Pat. No. 8,012,907B2), also required high temperature for successful crosslinking or surface crosslinking.

Thus, there is an urgent need in the art for a SAP that is both bio-based and biodegradable, while also being manufacturable under milder conditions without the need for high-temperature crosslinking processes.

### SUMMARY OF THE INVENTION

Accordingly, the present invention is to provide a formulation of a bio-based and bio-degradable SAP and a method of preparing the bio-based and bio-degradable SAP. The bio-based and bio-degradable SAP contains at least 80% of bio-based content according to an international standard test of ASTM D6866 conducted by a third party testing laboratory and can pass EN13432 industrial composting test, OECD 301B ready biodegradability test, REACH SVHC analysis, ISO 10993-10:2021 test for skin sensitization and ISO 10993-23:2021 test for irritation.

The bio-based and biodegradable SAP of the present invention is prepared via crosslinking reactions at temperatures below 80° C., thereby reducing energy consumption and enabling milder processing conditions compared to conventional petroleum-based SAPs and other prior arts of bio-based SAP.

In accordance with a first aspect of the present invention, the present invention provides a formulation of a bio-based and bio-degradable SAP. The bio-based and bio-degradable SAP includes sodium alginate (SA), modified cellulose, at least one activation agent, at least one crosslinking agent and at least one pH-adjusting agent. The bio-based and biodegradable superabsorbent polymer has a bio-based content from 0.1% to 93% and fulfills as least one bio-degradation regulation selected from EN 13432, ASTM D6400, OECD 301B, ISO 17088, EN 14995 or AS 5810.

In accordance to one embodiment, the bio-based and biodegradable superabsorbent polymer has a bio-based content from 20% to 93%.

In accordance to one embodiment, the modified cellulose includes carboxymethyl cellulose (CMC), carboxymethyl guar (CMG), hydroxyethyl cellulose (HEC), hydroxypropyl cellulose (HPC), carboxymethyl starch (CMS), or a combination thereof, and is present in an amount of 25 wt % to 85 wt %.

In accordance to one embodiment, the at least one activation agent includes a core activation agent or a surface activation agent.

In accordance to one embodiment, the at least one activation agent includes 1-ethyl-3-(3-dimethylaminopropyl)carbodiimide hydrochloride, dicyclohexylcarbodiimide, N-hydroxysulfosuccinimide, or a combination thereof, and is operable under pH 4 to 7 and ambient temperature conditions.

In accordance to one embodiment, the at least one crosslinking agent includes a core crosslinking agent or a surface crosslinking agent.

In accordance to one embodiment, the core crosslinking agent includes gelatin, chitosan, diethylenetriamine, propane-1,2,3-triamine, polyethylenimine, or a combination thereof; and the surface crosslinking agent comprises diethylenetriamine, propane-1,2,3-triamine, tris(2-aminoethyl)amine, polyetheramines having at least two terminal primary amine groups and a polypropylene glycol or polyethylene glycol or poly(oxypropylene-co-oxyethylene) backbone with molecular weights ranging from approximately 400 to 5000 g/mol, or a combination thereof.

In accordance to one embodiment, the at least one pH-adjusting agent comprises hydrochloric acid solution, acetic acid solution, phosphoric acid solution, nitric acid solution, ammonium solution, potassium hydroxide, potassium hydroxide solution, sodium hydroxide, sodium hydroxide solution, or a combination thereof.

In accordance to another embodiment, the bio-based and biodegradable superabsorbent polymer further includes at least one surfactant, itaconic acid (IA), acrylamide, at least one radical initiator and at least one di-acrylate.

In accordance to one embodiment, the at least one surfactant includes octylphenol ethoxylates having an average ethylene oxide chain length of approximately 7 to 70 ethylene oxide units, alkyl polyglucosides comprising coco glucoside, decyl glucoside, octyl glucoside, lauryl glucoside, sucrose cocoate, C₆ alkyl glycoside, C₈₋₁₀ alkyl glycoside, or a combination thereof, and is present in an amount of 0.1 wt % to 10 wt %.

In accordance to one embodiment, the at least one radical initiator comprises tert-butyl hydroperoxide, benzoyl peroxide, ammonium persulfate, di-tert-butyl peroxide, potassium persulfate, or a combination thereof.

In accordance to one embodiment, the at least one di-acrylate includes poly(propylene glycol) diacrylate, poly(ethylene glycol) diacrylate, N,N′-methylenebis(acrylamide), tri(ethylene glycol) diacrylate, 1,6-hexanediol diacrylate, or a combination thereof.

In accordance to one embodiment, the bio-based and biodegradable superabsorbent polymer is formed into granules with a particle size of 20-100 mesh, and exhibits a free swelling capacity (FSC) of at least 36 g/g saline solution, a centrifuge retention capacity (CRC) of at least 24 g/g saline solution, and an absorption under load (AUL) of at least 8 g/g saline solution. The SAP granules pass ISO 10993-10:2021 test for skin sensitization, ISO 10993-23:2021 test for irritation and REACH SVHC analysis.

In a second aspect, the present invention provides a method for preparing a bio-based and biodegradable superabsorbent polymer. The method includes dissolving sodium alginate and modified cellulose with weight ratio of 1:0.3-6 in DI water to form the polysaccharide solution; core crosslinking of the polysaccharide solution to form a core-crosslinked gel; drying the core-crosslinked gel at 50° C. to 110° C. to form a core superabsorbent polymer; milling and sieving the core superabsorbent polymer to form one or more core superabsorbent polymer granules with a particle size with mesh size smaller than 100 mesh; surface crosslinking of the one or more core superabsorbent polymer granules to form the one or more superabsorbent polymer granules; and drying the one or more superabsorbent polymer granules to form the bio-based and biodegradable superabsorbent polymer. The bio-based and biodegradable superabsorbent polymer exhibits a bio-based content greater than 80%, a free swell capacity of at least 36 g/g saline solution, and undergoes at least 90% degradation within 180 days in accordance with the EN 13432 standard or at least 60% degradation within 28 days in accordance with the OECD 301B standard. The entire process avoids temperatures exceeding 110° C., uses no petroleum-derived acrylic acid, and yields a compostable SAP with no phytotoxicity.

In accordance to one embodiment, the step of dissolving sodium alginate and modified cellulose in DI water to form the polysaccharide solution further includes adding at least one surfactant, itaconic acid, acrylamide, at least one radical initiator and at least one di-acrylate into the polysaccharide solution.

In accordance to one embodiment, the itaconic acid is further subjected to radical polymerization to form a polymer of itaconic acid, and the polymerization of the itaconic acid is conducted at a temperature of 50° C. to 90° C. under nitrogen, optionally with acrylamide in a molar ratio of 3-20:1 and one or more di-acrylates.

In accordance to one embodiment, the step of dissolving sodium alginate and modified cellulose in DI water to form the polysaccharide solution including: adding and stirring the sodium alginate and the modified cellulose in the DI water until fully dissolved; and subsequently adding at least one pH-adjusting agent while stirring to adjust the pH of the polysaccharide solution to a range of 4 to 7.

In accordance to one embodiment, the step of core crosslinking of the polysaccharide solution to form a core-crosslinked gel including:
adding at least one core activation agent to the polysaccharide solution;
placing the polysaccharide solution at room temperature to allow core activation;
dissolving at least one core crosslinking agent in DI water to form a core crosslinking agent solution;
mixing the core crosslinking agent solution with the polysaccharide solution to form a mixture;
adding at least one pH-adjusting agent while stirring to adjust the pH of the mixture to a range of 7 to 12 and placing the mixture at room temperature to allow core crosslinking to form the core-crosslinked gel.

In accordance to one embodiment, the step of surface crosslinking of the one or more core superabsorbent polymer granules including:
stirring the core superabsorbent polymer granules in at least one solvent at room temperature;
adding at least one surface activation agent to the core superabsorbent polymer granules while stirring at room temperature to allow surface activation;
adding at least one surface crosslinking agent to the core superabsorbent polymer granules while stirring at room temperature to allow surface crosslinking to form the superabsorbent polymer granules; and
washing the superabsorbent polymer granules with the at least one solvent.

In accordance to one embodiment, the step of drying the one or more superabsorbent polymer granules to form the bio-based and biodegradable superabsorbent polymer includes drying the one or more superabsorbent polymer granules at 50° C. to 110° C.

### BRIEF DESCRIPTION OF THE DRAWINGS

Embodiments of the invention are described in more details hereinafter with reference to the drawings, in which:

FIG. 1 schematically depicts the core activation reaction between a carboxyl group from sodium alginate or modified cellulose and the at least one activation agent, and the core crosslinking reaction between the activated carboxyl group and an amine group of a core crosslinking agent;

FIG. 2 schematically depicts the surface activation reaction between a carboxyl group on the surface of the core SAP granules and the at least one activation agent, and the surface crosslinking reaction between the activated carboxyl group and an amine group of a surface crosslinking agent;

FIGS. 3A-3B depict the radical polymerization of IA alone and IA together with acrylamide in certain embodiments;

FIG. 4A depicts bio-based carbon content of core SAP granules from Example 3 measured by ASTM D6866 and FIG. 4B depicts bio-based carbon content of SAP granules from Example 8 measured by ASTM D6866;

FIG. 5 depicts the main results of EN 13432 industrial composting test of a certain embodiment of the bio-based and biodegradable SAP of the present invention. The test is determined by a third party testing laboratory; and

FIG. 6 depicts the main results of OECD 301B ready biodegradability test of a certain embodiment of the bio-based and biodegradable SAP of the present invention. The test is determined by a third party testing laboratory.

### DETAILED DESCRIPTION

### Definitions

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they allow for elements not explicitly recited, but exclude elements that are found in the prior art or that affect a basic or novel characteristic of the present invention.

Furthermore, throughout the specification and claims, unless the context requires otherwise, the word “include” or variations such as “includes” or “including”, will be understood to imply the inclusion of a stated integer or group of integers but not the exclusion of any other integer or group of integers.

As used herein and not otherwise defined, the terms “substantially,” “substantial,” “approximately” and “about” are used to describe and account for small variations. When used in conjunction with an event or circumstance, the terms can encompass instances in which the event or circumstance occurs precisely as well as instances in which the event or circumstance occurs to a close approximation. For example, when used in conjunction with a numerical value, the terms can encompass a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

References in the specification to “one embodiment”, “an embodiment”, “an example embodiment”, etc., indicate that the embodiment described can include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

In the methods of preparation described herein, the steps can be carried out in any order without departing from the principles of the invention, except when a temporal or operational sequence is explicitly recited. Recitation in a claim to the effect that first a step is performed, and then several other steps are subsequently performed, shall be taken to mean that the first step is performed before any of the other steps, but the other steps can be performed in any suitable sequence, unless a sequence is further recited within the other steps. For example, claim elements that recite “Step A, Step B, Step C, Step D, and Step E” shall be construed to mean step A is carried out first, step E is carried out last, and steps B, C, and D can be carried out in any sequence between steps A and E, and that the sequence still falls within the literal scope of the claimed process. A given step or sub-set of steps can also be repeated. Furthermore, specified steps can be carried out concurrently unless explicit claim language recites that they be carried out separately.

As used herein, the term “bio-based content” refers to the proportion of organic carbon content in a material that is derived from renewable biological sources, measured according to ASTM D6866 standard by a certified third-party testing laboratory.

As used herein, the term “biodegradation regulation” refers to a standard protocol that assesses the biodegradability of materials, including but not limited to EN 13432, ASTM D6400, OEDC 301B, ISO 17088, EN 14995 and AS 5810, wherein biodegradation is confirmed if the material meets the threshold criteria specified in the corresponding standard.

As used herein, the term “modified cellulose” refers to cellulose that has been chemically altered to incorporate functional groups.

As used herein, the term “activation agent” refers to a chemical reagent that modifies carboxyl groups on polysaccharides to generate reactive intermediates capable of undergoing further crosslinking reactions with amine groups, and includes both core activation agents and surface activation agents.

As used herein, the term “crosslinking agent” refers to a molecule or polymer containing at least two reactive functional groups, such as amine groups, that react with activated polysaccharides to form covalent bonds, thereby creating a three-dimensional network structure within the superabsorbent polymer.

As used herein, the terms “core activation agent” and “surface activation agent” refer respectively to activation agents used to activate carboxyl groups in the bulk matrix or on the surface of SAP granules for subsequent crosslinking reactions.

As used herein, the terms “core crosslinking agent” and “surface crosslinking agent” refer respectively to crosslinking agents that react with the activated functional groups within the bulk matrix or at the surface of the superabsorbent polymer granules to form covalent bonds and stabilize the polymer structure.

As used herein, the term “pH-adjusting agent” refers to an acid or base, including but not limited to hydrochloric acid, acetic acid, sodium hydroxide, or ammonium solution, that is added to adjust the pH of a solution to a desired range to facilitate chemical reactions.

As used herein, the term “superabsorbent polymer granules” refers to discrete particles of crosslinked polymer material having a particle size between 20 mesh and 100 mesh, capable of absorbing and retaining large amounts of liquid relative to their mass without dissolving.

As used herein, “free swelling capacity (FSC)” refers to the amount of liquid absorbed by a dry SAP sample under free swelling conditions; “centrifuge retention capacity (CRC)” refers to the amount of liquid retained in the SAP after centrifugation under a specified centrifugal force; and “absorption under load (AUL)” refers to the amount of liquid absorbed by a SAP sample under a specified mechanical load. The measurements are conducted using saline solution unless otherwise specified. In the present invention, the FSC, CRC and AUL of the SAP was measured by Edana test methods 2002 440.2-02, 441.2-02 and 442.2-02 testing methods, respectively.

As used herein, the biodegradation standards EN 13432, ASTM D6400, OECD 301B, ISO 17088, EN 14995 and AS 5810 are each incorporated by reference in their entirety. These standards may be periodically updated; therefore, any reference to such standards is intended to encompass all applicable versions, unless otherwise stated.

The term “mesh size” refers to the number of openings per inch in the sieve. A larger mesh number corresponds to smaller openings and thus smaller particles, while a smaller mesh number corresponds to larger openings and larger particles.

Other definitions for selected terms used herein may be found within the detailed description of the present invention and apply throughout. Unless otherwise defined, all other technical terms used herein have the same meaning as commonly understood to one of ordinary skill in the art to which the present invention belongs.

In the following description, the formulations, compositions and methods for producing and using the same, and the likes, are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

The present invention proposes a new SAP formulation which has a high content of bio-based raw materials and the preparation method for preparing the same. The bio-based and bio-degradable SAP includes sodium alginate (SA), modified cellulose, at least one activation agent, at least one crosslinking agent and at least one pH-adjusting agent. SA provides both a high molecular weight to form the framework of the SAP for high volume expansion and carboxyl group for crosslinking, especially core crosslinking. The modified cellulose provides both a high molecular weight to form the framework of the SAP for high volume expansion and carboxyl group for crosslinking, especially surface crosslinking. The pH-adjustment agent is added to the polysaccharide solution to adjust the pH of the polysaccharide solution to pH 4-7, preferably pH 5, in order to protonate the carboxyl group of SA and modified cellulose.

In an embodiment of the first aspect, the bio-based and bio-degradable SAP contains 10-70% by weight of SA. Preferably, the SA has a viscosity of 100-4000 cP at room temperature.

In an embodiment of the first aspect, the bio-based and bio-degradable SAP contains 25-85% by weight of modified cellulose. The modified cellulose may include carboxymethyl cellulose (CMC), carboxymethyl guar (CMG), hydroxyethyl cellulose (HEC), hydroxypropyl cellulose (HPC), carboxymethyl starch (CMS), or a combination thereof.

Preferably, the modified cellulose has a viscosity of 500-5000 cP at room temperature.

In an embodiment of the first aspect, the bio-based and bio-degradable SAP contains 1-15% by weight of at least one activation agent. The at least one activation agent includes core activation agent or surface activation agent.

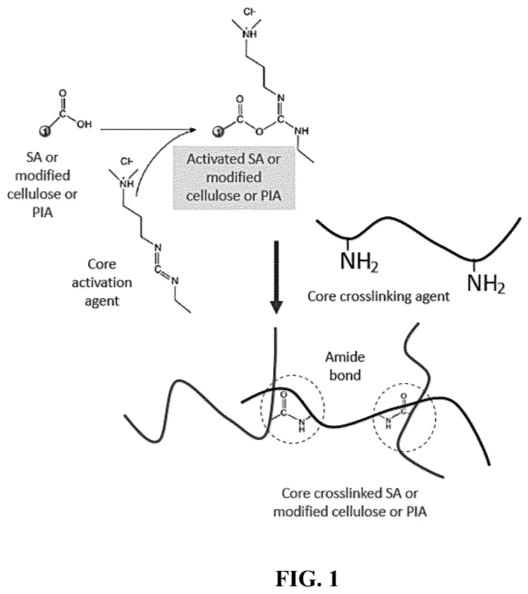

The core activation agent is reacted with a small portion of the carboxyl groups on the SA and modified cellulose chains to form the covalently activated SA and activated modified cellulose. The activated SA and modified cellulose are semi-stable at room temperature and can further react with a crosslinking agent. The activation agent reacts with the protonated carboxyl group. Therefore, the activation reaction is conducted in acid to neutral condition and the reaction can be conducted in mild temperature, such as room temperature. FIG. 1 schematically depicts the core crosslinking reaction between a carboxyl group from sodium alginate or modified cellulose and an amine group from core crosslinking agent in certain embodiment.

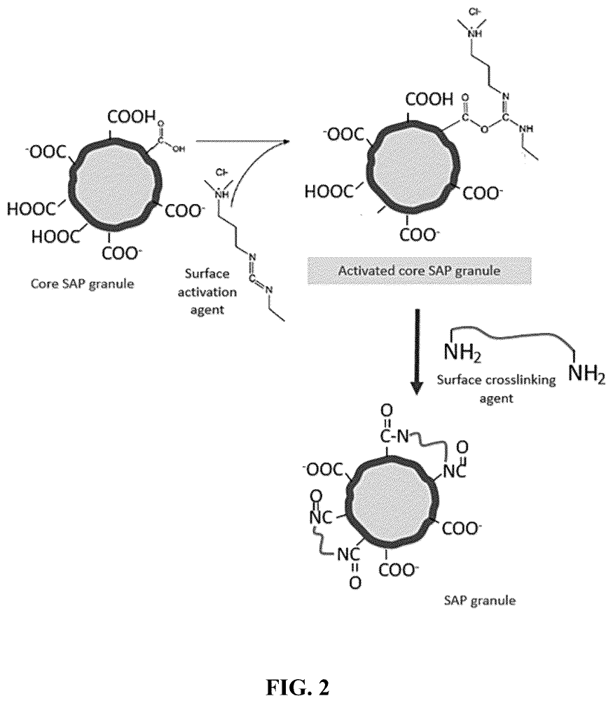

The surface activation agent is reacted with a small portion of the carboxyl groups on the core SAP granules to form the covalently activated core SAP granules. The activated core SAP granules are semi-stable at room temperature and can further react with a surface crosslinking agent. The reaction can be conducted in mild temperature, such as room temperature. FIG. 2 schematically depicts the surface crosslinking reaction between a carboxyl group on the surface of the core SAP granules and an amine group from surface crosslinking agent in certain embodiment.

Preferably, the at least one activation agent may include 1-ethyl-3-(3-dimethylaminopropyl)carbodiimide hydrochloride, dicyclohexylcarbodiimide, N-hydroxysulfosuccinimide, or a combination thereof.

The core crosslinking agent is amine-based molecules or polymers which contains at least two amine groups, which reacts with the activated SA and activated modified cellulose to form an amide bond between SA and modified cellulose by nucleophilic attack. The crosslinking agent reacts in non-protonated state. Therefore, the crosslinking reaction is conducted in neutral or alkaline condition and the reaction can be conducted in mild temperature, such as room temperature.

In an embodiment of the first aspect, the bio-based and bio-degradable SAP contains 0.1-25% by weight of at least one crosslinking agent. The at least one crosslinking agent includes core crosslinking agent or surface crosslinking agent. The core crosslinking agent may include gelatin, chitosan, diethylenetriamine, propane-1,2,3-triamine, polyethylenimine, or a combination thereof. The surface crosslinking agent may include diethylenetriamine, propane-1,2,3-triamine, tris(2-aminoethyl)amine, polyetheramines, Jeffamine® T-403, T-5000, T-3000, ED-600, ED-900, ED-2003, or a combination thereof.

In certain embodiments, the surface crosslinking agent is amine-based molecule(s) or polymer(s) which contain(s) at least two amine groups, which react with the activated core SAP granules to form amide bond on the surface of the core SAP granules. The reaction can be conducted in mild temperature, such as room temperature.

The SAP granules are washed with at least one solvent, which is a combination of at least two of DI water, methanol, ethanol, propanol or acetone, after surface crosslinking reaction to remove impurity. Water hydrolyzes the unreacted activated core SAP granules and re-generates COOH groups while methanol, ethanol, propanol or acetone control the degree of swelling to minimize water absorption of the SAP during washing.

In certain embodiments, the pH-adjustment agent is added after adding the core crosslinking agent to adjust the pH of the core crosslinking agent to pH 7-10 in order to facilitate nucleophilic substitution of amine group of the activation agent to form covalent bond with the activated SA and activated modified cellulose.

In an embodiment of the first aspect, the at least one pH-adjusting agent may include hydrochloric acid solution, acetic acid solution, phosphoric acid solution, nitric acid solution, ammonium solution, potassium hydroxide, potassium hydroxide solution, sodium hydroxide, sodium hydroxide solution, or a combination thereof.

Optionally, the bio-based and bio-degradable SAP includes at least one surfactant, itaconic acid (IA), acrylamide, at least one radical initiator and at least one di-acrylate.

In an embodiment of the first aspect, the surfactant is used for pore forming or stabilization to create or stabilize small pores within SAP granules or to increase surface area of the SAP in order to increase liquid absorption speed by capillary action. The at least one surfactant may include Triton X-100, X-102, X-114, X-165, X-305, X-705, coco glucoside, decyl glucoside, octyl glucoside, lauryl glucoside, sucrose cocoate, C₆ alkyl glycoside, C₈₋₁₀ alkyl glycoside, or a combination thereof.

In an embodiment of the first aspect, the bio-based and bio-degradable SAP contains 0-50% by weight of IA. Preferably, IA is derived from renewable plant sources, i.e. bio-based chemical.

In another embodiment of the first aspect, the bio-based and bio-degradable SAP contains 0-10% by weight of acrylamide.

In an embodiment of the first aspect, the radical initiator is used for initiating the radical polymerization. It decomposes at the preferred temperature to form active radical, which attacks the C═C double bond of IA (and acrylamide) to transfer the radical site to IA (or acrylamide). The radical containing IA further attacks remaining IA (or acrylamide) for chain propagation to form PIA with preferred molecular weight and monomer conversion. The at least one radical initiator may include tert-butyl hydroperoxide, benzoyl peroxide, ammonium persulfate, di-tert-butyl peroxide, potassium persulfate, or a combination thereof.

In an embodiment of the first aspect, the di-acrylate is the crosslinking agent of radical polymerization which contains two C═C double bond to join two polymer chains of IA (and acrylamide) together to form a network of resultant PIA. The at least one di-acrylate may include poly(propylene glycol) diacrylate, poly(ethylene glycol) diacrylate, N,N′-methylenebis(acrylamide), tri(ethylene glycol) diacrylate, 1,6-hexanediol diacrylate, or a combination thereof.

In an embodiment of the first aspect, the bio-based and bio-degradable SAP has a bio-based content from 0.1% to 93%.

In certain embodiments, nitrogen gas is used to expel oxygen dissolved in the IA solution as oxygen can terminate the radical polymerization.

After core crosslinking and drying, the core SAP granules are milled into granules with suitable particle size, preferably between 20-100 mesh.

The present invention also exhibits excellent and verified bio-degradability and safety to human use. Such a SAP is derived predominantly from renewable resources, exhibits high biodegradability in accordance with international standards, and utilizes environmentally friendly crosslinking agents that enable efficient gel formation at lower temperatures. This would address the growing market demand for sustainable alternatives to petroleum-based SAPs, reduce the environmental footprint of disposable hygiene products, and align with global efforts toward eco-friendly material innovation.

In accordance with a second aspect of the present invention, there is provided a method of preparing the bio-based and bio-degradable SAP. The method for preparing the bio-based and bio-degradable SAP comprising:
(1) dissolving SA and modified cellulose in DI water to form the polysaccharide solution;
(2) core-crosslinking of the polysaccharide solution to form the core-crosslinked gel;
(3) drying the core-crosslinked gel to form the core SAP;
(4) milling the core SAP to form the core SAP granules;
(5) surface crosslinking of the core SAP granules to form the SAP granules; and
(6) drying the SAP granules.

The crosslinking and surface crosslinking methods used are mild and effective that successful crosslinking can take place even at room temperature to give large capacity to absorb water, saline solutions, biological fluids, and the like, at normal pressure or under load, and to retain these fluids.

Optionally, the method for preparing the bio-based and bio-degradable SAP further includes radical polymerization of IA to form the polymer of itaconic acid (PIA).

In an embodiment of the second aspect, the step of dissolving SA and modified cellulose in DI water to form the polysaccharide solution comprising: 1) adding and stirring SA and modified cellulose in DI water until they are completely dissolved and 2) adding at least one pH-adjusting agent with stirring to adjust the pH of the solution to 4-7.

In an embodiment of the second aspect, the step of core crosslinking of the polysaccharide solution to form the core-crosslinked gel comprising:
(1) adding at least one core activation agent to the polysaccharide solution with stirring;
(2) placing the polysaccharide solution at room temperature for 15 minutes to 2 hours to allow core activation;
(3) dissolving at least one core crosslinking agent in DI water;
(4) adding the core crosslinking agent solution into the polysaccharide solution with stirring;
(5) adding at least one pH-adjusting agent with stirring to adjust the pH of the solution to 7-12; and
(6) placing the polysaccharide solution at room temperature for 15 minutes to 2 hours to allow core crosslinking to form the core-crosslinked gel.

Optionally, the method includes adding at least one surfactant into the polysaccharide solution with stirring.

Optionally, the method includes adding the PIA to the polysaccharide solution with stirring. PIA contains a high concentration of anionic carboxyl groups (two carboxyl groups per monomer unit) in its polymer chain. This provides a large charge repulsion when it is in contact with aqueous media.

In certain embodiments, the PIA is synthesized by radical polymerization of IA (and acrylamide). The acrylamide is added to have radical polymerization together with IA because the reactivity of acrylamide is higher than that of IA and the resultant molecular weight is higher than IA alone. Preferably, the mole ratio of IA-to-acrylamide is 3-20:1.

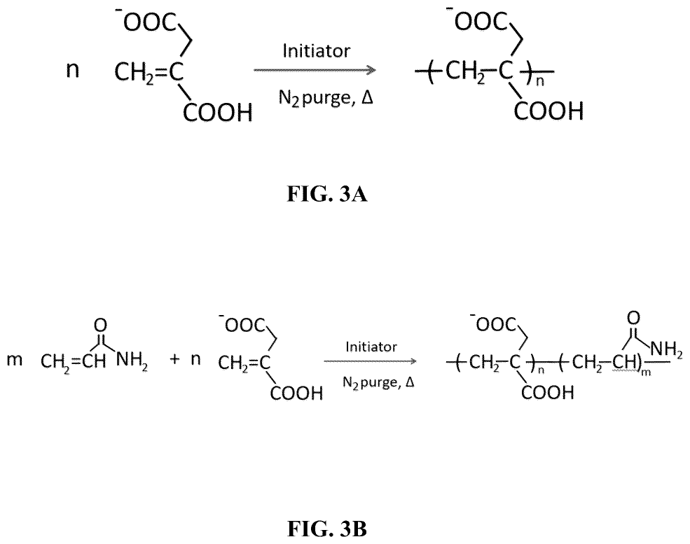

Preferably, the IA is produced by fermentation of plant-based feedstock, such as cornstarch. The radical polymerization of IA alone or together with acrylamide is illustrated in FIGS. 3A-3B.

In an embodiment of the second aspect, the step of drying of the core-crosslinked gel to form the core SAP including drying core-crosslinked gel in 50-110° C. oven.

In an embodiment of the second aspect, the step of milling of the core SAP to form the core SAP granules including: 1) milling the core SAP into granule form and 2) sieving the core SAP granules to remove the core SAP granules with mesh size larger than 100 mesh.

In an embodiment of the second aspect, the step of surface crosslinking of the core SAP granules to form the SAP granules including:
(1) stirring the core SAP granules in at least one solvent at room temperature for 1-55 minutes;
(2) adding at least one surface activation agent to the core SAP granules with stirring for 1-55 minutes at room temperature to allow surface activation;
(3) adding at least one surface crosslinking agent to the core SAP granules with stirring for 0.5-3 hours at room temperature to allow surface crosslinking to form the SAP granules; and
(4) washing the SAP granules with at least one solvent.

In an embodiment of the second aspect, the step of drying of the SAP granules including drying the SAP granules in 50-110° C. oven.

In an embodiment of the second aspect, the step of radical polymerization of itaconic acid to form the PIA including:
(1) adding and stirring IA in DI water;
(2) adding at least one pH-adjusting agent with stirring to partially neutralize IA;
(3) purging nitrogen gas to the stirring mixture for 10-40 minutes;
(4) increasing the temperature of the stirring mixture to 50-90° C. with continuous nitrogen gas purging;
(5) dissolving at least one radical initiator into DI water and add the at least one radical initiator solution into the stirring mixture to start polymerization;
(6) stirring at 50-90° C. for further 2-6 hours with nitrogen gas purging to form the PIA solid;
(7) cutting the PIA solid into small pieces;
(8) drying PIA solid in 50-110° C. oven; and
(9) milling the PIA solid to form the PIA.

Optionally, the method further includes adding acrylamide into the stirring mixture.

Optionally, the method further includes adding at least one di-acrylate after 5-60 mins of reaction.

Optionally, the method further includes adding at least one pH-adjusting agent to further neutralize PIA solid.

In an embodiment of the second aspect, the step of radical polymerization of IA to form the PIA is before the step of dissolving SA and modified cellulose in DI water to form the polysaccharide solution.

In an embodiment of the second aspect, the modified cellulose may include CMC, CMG, HEC, HPC, CMS, or a combination thereof.

In an embodiment of the second aspect, the at least one core activation agent may include 1-ethyl-3-(3-dimethylaminopropyl)carbodiimide hydrochloride, dicyclohexylcarbodiimide, N-hydroxysulfosuccinimide, or a combination thereof.

In an embodiment of the second aspect, the at least one core crosslinking agent may include gelatin, chitosan, diethylenetriamine, propane-1,2,3-triamine, polyethylenimine, or a combination thereof.

In an embodiment of the second aspect, the at least one surface crosslinking agent may include diethylenetriamine, propane-1,2,3-triamine, tris(2-aminoethyl)amine, polyetheramines, Jeffamine® T-403, T-5000, T-3000, ED-600, ED-900, ED-2003, or a combination thereof.

In an embodiment of the second aspect, the at least one pH-adjusting agent may include hydrochloric acid solution, acetic acid solution, phosphoric acid solution, nitric acid solution, ammonium solution, potassium hydroxide, potassium hydroxide solution, sodium hydroxide, sodium hydroxide solution, or a combination thereof.

In an embodiment of the second aspect, the at least one solvent may include DI water, methanol, ethanol, propanol, acetone, or a combination thereof.

In an embodiment of the second aspect, the at least one surfactant may include Triton X-100, X-102, X-114, X-165, X-305, X-705, coco glucoside, decyl glucoside, octyl glucoside, lauryl glucoside, sucrose cocoate, C₆ alkyl glycoside, C₈₋₁₀ alkyl glycoside, or a combination thereof.

In an embodiment of the second aspect, IA is derived from renewable plant sources, i.e. bio-based chemical.

In an embodiment of the second aspect, the at least one radical initiator may include tert-butyl hydroperoxide, benzoyl peroxide, ammonium persulfate, di-tert-butyl peroxide, potassium persulfate, or a combination thereof.

In an embodiment of the second aspect, the at least one di-acrylate may include poly(propylene glycol) diacrylate, poly(ethylene glycol) diacrylate, N,N′-methylenebis(acrylamide), tri(ethylene glycol) diacrylate, 1,6-hexanediol diacrylate, or a combination thereof.

In the following description, specific details are provided to offer a comprehensive understanding of the present invention, for explanatory purposes and not intended for limitation.

### EXAMPLE

### Example 1—Synthesis of PIA with IA Alone

50 g IA and 25 g DI water was mixed in a round bottom flask with magnetic stirring. 15 g of sodium hydroxide was added to the mixture slowly to dissolve the solid. Then the solution mixture was purged with nitrogen for 30 mins and the flask was placed in an oil bath with magnetic stirring equipped with a condenser. The temperature of the oil bath was increased to 80° C. 0.25 g of ammonium persulfate was dissolved in 1 mL DI water and the solution was added to the IA solution dropwisely to initiate polymerization. The mixture was kept at 80° C. for 4 hours with stirring. After that, the solid product was taken out. The PIA solid was cut into small pieces followed by milling into granules. The PIA was 50% neutralized.

For measurement of monomer conversion, the PIA solution was freeze dried and re-dissolved in deuterium dioxide. The solution was measured with proton nuclear magnetic resonance (NMR). The monomer conversion was calculated by the ratio of proton integration of monomer IA and polymer PIA. The monomer conversion is between 88-99%.

The molecular weight of the PIA was measured by gel permeation chromatography with pH 7 phosphate buffered saline (PBS) solution as mobile phase and polyacrylic acid standards. The number average molecular weight is between 22 k-34 k g/mol.

### Example 2—Synthesis of PIA with IA, Acrylamide and Di-Acrylate

50 g IA and 31 g DI water was mixed in a plastic bottle with cap with magnetic stirring. 15 g of sodium hydroxide was added to the mixture slowly to dissolve the solid. Then the solution mixture was purged with nitrogen for 20 mins and the flask was placed in a water bath with magnetic stirring. 8 g acrylamide was added to the mixture. The temperature of the water bath was increased to 70° C. 0.25 g of ammonium persulfate was dissolved in 0.5 mL DI water and the solution was added to the IA solution dropwisely to initiate polymerization. 0.313 g of polyethylene glycol diacrylate was added to the mixture after 10 mins. The mixture was kept at 70° C. for 4 hours with stirring. After that, the solid product was taken out. The PIA solid was cut into small pieces followed by milling into granules. The PIA was 50% neutralized.

To obtained PIA with 70% neutralization, the PIA solid was cut into small pieces and placed in 810 mL of 0.2M sodium hydroxide solution for absorption. The PIA gel was dried in 70° C. oven and the solid was milled into granules. The PIA was 70% neutralized.

### Example 3—Formation of Core SAP Granules with PIA and SA-Diethylenetriamine as Core Crosslinking Agent

SA was dissolved in DI water to form a 4% solution. The pH of the SA solution was adjusted to 5 using NaOH solution. 1-Ethyl-3-(3-dimethylaminopropyl)carbodiimide hydrochloride (EDC) was added and dissolved into the SA solution in a 10% weight ratio of SA. The SA/EDC mixture was allowed to have core activation reaction at room temperature for 1 hour. The PIA (IA alone) was dissolved in DI water and the solution was adjusted to pH 8 using NaOH solution. The two solutions were then mixed together with mass ratio of PIA-to-SA of 1:3. After that diethylenetriamine was mixed into the mixture in a 25% mole ratio of EDC. The mixture was placed at room temperature for another 1 hour for core crosslinking reaction. After that, the mixture was then placed in a 70° C. oven for drying for 2-4 hours.

The dried solid was milled into granules using a centrifuge mill. The core SAP granules were classified based on their particle size by sieving method. The Free swelling capacity (FSC), centrifuge retention capacity (CRC) and absorption under load (AUL) of the SAP were measured. The FSC, CRC and AUL were 47, 40 and 7.3 g saline/g of SAP, respectively.

The bio-degradation of the SAP was measured according to EN 13432 industrial composting standard and the measurement was conducted by a third party testing laboratory. The SAP showed 90.6% degradation during 90 days according to the EN 13432 regulation, 93.2% disintegration after 84 days and did not have any observable phytotoxic effect according to OECD 208 modified Annex E of EN 13432 test. The CO₂ evolution analysis demonstrated that the cumulative carbon dioxide released from the SAP reached more than 90% of the theoretical maximum within 180 days, thereby satisfying the biodegradability requirement of EN 13432. Disintegration analysis confirmed that over 93% of the SAP granules were physically broken down to particles smaller than 2 mm after 84 days of composting. Furthermore, phytotoxicity assessment using cress seed germination and biomass analysis revealed no significant growth inhibition compared to the control group, indicating the absence of phytotoxic effects. These combined results confirm that the SAP granules fulfill all key criteria for industrial compostability.

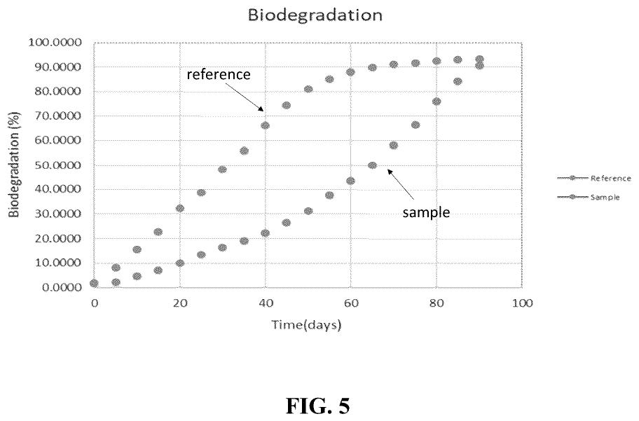

FIG. 5 illustrates the main experimental results of the EN 13432 industrial composting test of the bio-based and biodegradable SAP of the present invention. It includes three sub-sections: (1) biodegradation percentage as a function of composting time, showing CO₂ evolution exceeding 90% within 90 days; (2) disintegration rate, confirming that more than 93% of the SAP fragments passed through a 2 mm sieve after 84 days; and (3) phytotoxicity testing results based on OECD 208 seed germination and biomass analysis, which demonstrated no toxic effects on plant growth. These results visually corroborate the SAP's compliance with industrial composting standards, supporting its environmental friendliness and applicability in compostable hygiene or agricultural products.

### Example 4—Formation of SAP Granules—Diethylenetriamine as Surface Crosslinking Agent

The core SAP granules of Example 3 were immersed and stirred in acetone/water (9:1 v/v) solution for 10 mins. After that, EDC was added to the acetone/water solution in 2.5% weight ratio of core SAP granules with continuous stirring at room temperature for 20 mins for surface activation reaction. Then diethylenetriamine was added into the mixture in a 25% mole ratio of EDC followed by continuous stirring at room temperature for 20 mins for surface crosslinking reaction. The SAP granules were separated from the solution and were washed with acetone. The SAP granules was dried in 60° C. oven for 30 mins. The FSC, CRC and AUL were 36, 24 and 12.2 g saline/g of SAP, respectively.

### Example 5—Formation of Core SAP Granules with SA and Modified Cellulose-Gelatin as Core Crosslinking Agent

SA and CMC, with weight ratio of 1:1, were dissolved together in DI water to form a 3.8% solution. The pH of the solution was adjusted to 5 using NaOH solution. EDC was added and dissolved into the solution in a 1.75% weight ratio of SA and CMC. The mixture was allowed to have core activation reaction at room temperature for 1 hour. After that gelatin was dissolved in DI water and was added into the mixture in a 20% weight ratio of SA. NaOH solution was added to the mixture to deprotonate the amine groups of gelatin. The mixture was placed at room temperature for another 1 hour for core crosslinking reaction. After that, the mixture was then placed in a 70° C. oven for drying for 4-6 hours.

The dried solid was milled into granules using a centrifuge mill. The core SAP granules were classified based on their particle size by sieving method. The FSC and CRC were 58 and 22 g saline/g of SAP, respectively.

### Example 6—Formation of SAP Granules with SA and Modified Cellulose-Gelatin as Core Crosslinking Agent and Diethylenetriamine as Surface Crosslinking Agent

SA and CMC, with weight ratio of 1:3, were dissolved together in DI water to form a 3.5% solution. The pH of the solution was adjusted to 5 using NaOH solution. EDC was added and dissolved into the solution in a 5% weight ratio of SA and CMC. The mixture was allowed to have core activation reaction at room temperature for 1 hour. After that gelatin was dissolved in DI water and was added into the mixture in a 25% weight ratio of SA and CMC. NaOH solution was added to the mixture to deprotonate the amine groups of gelatin. The mixture was placed at room temperature for another 1 hour for core crosslinking reaction. After that, the mixture was then placed in a 70° C. oven for drying for 4-6 hours. The dried solid was milled into granules using a centrifuge mill. The core SAP granules were classified based on their particle size by sieving method.

The core SAP granules between 20-60 mesh were immersed and stirred in acetone/water (9:1 v/v) solution for 10 mins. After that, EDC was added to the acetone/water solution in 7% weigh ratio of the core SAP granules with continuous stirring at room temperature for 15 mins for surface activation reaction. Then diethylenetriamine was added into the mixture in a 4% mole ratio of EDC followed by continuous stirring at room temperature for 40 mins for surface crosslinking reaction. The SAP granules were separated from the solution and were washed with ethanol/water (7:3 v/v) solution. The SAP granules was dried in 70° C. oven for 30 mins. The FSC, CRC and AUL were 61, 46 and 11.3 g saline/g of SAP, respectively.

The absorption speed of the SAP granules was measured by vortex method. By which, 25 mL of saline solution was magnetically stirred using a magnetic stirrer bar (2.5 cm long and 0.5 cm diameter) with 600 rpm speed in a 50 mL beaker. A vortex was created. 1.0 g of the SAP granules were poured into the saline solution and the SAP granules started absorbing the saline and caused disappearance of the vortex. The time between pouring the SAP granules and disappearance of the vortex was recorded as the absorption speed. The absorption speed of 20-60 mesh SAP granules was 67 seconds while that of 60-80 mesh SAP granules was 19.5 seconds.

The liquid permeability of the SAP granules was measured by column method. The SAP granules were immersed in excessive amount of saline for 1 hour at room temperature to allow swelling into gel. The SAP gel was placed into a glass column was packed to form a 2 cm thick gel layer. The SAP gel was covered by a metal mesh to immobilize the SAP gel at the bottom of the glass column. Saline solution was poured into the column and the height of the saline solution was kept at 9 cm height. The time for 20 mL of saline solution to pass through was measured. The liquid permeability of the SAP granules was calculated by the volume of saline passing through per minute. The liquid permeability of 20-60 mesh SAP granules was 6.0 mL/min and that of 60-80 mesh SAP granules was 10.7 mL/min.

The bio-based content of the SAP was measured by ASTM D6866 international standard test and the measurement was conducted by a third party testing laboratory. The bio-based content of the surface crosslinked SAP granules was 84.61%.

### Example 7—Formation of SAP Granules with SA, Modified Cellulose and Surfactant-Gelatin as Core Crosslinking Agent and Jeffamine® T5000 as Surface Crosslinking Agent

SA and CMC, with weight ratio of 1:3, were dissolved together in DI water to form a 3.5% solution. The pH of the solution was adjusted to 5 using NaOH solution. EDC was added and dissolved into the solution in a 5% weight percent of SA and CMC. The mixture was allowed to have core activation reaction at room temperature for 1 hour. After that gelatin was dissolved in DI water and was added into the mixture in a 25% weight ratio of SA and CMC. NaOH solution was added to the mixture to deprotonate the amine groups of gelatin. Decyl glucoside was added to the mixture in a 6.25% weight percent of SA and CMC. The mixture was placed at room temperature for another 1 hour for core crosslinking reaction. After that, the mixture was then placed in a 70° C. oven for drying for 4-6 hours. The dried solid was milled into granules using a centrifuge mill. The core SAP granules were classified based on their particle size by sieving method.

The core SAP granules between 20-60 mesh were immersed and stirred in acetone/water (9:1 v/v) solution for 10 mins. After that, EDC was added to the acetone/water solution in 10% weigh ratio of the core SAP granules with continuous stirring at room temperature for 15 mins for surface activation reaction. Then Jeffamine® T5000 was added into the mixture in a 4% mole ratio of EDC followed by continuous stirring at room temperature for 45 mins for surface crosslinking reaction. The SAP granules were separated from the solution and were washed with ethanol/water (7:3 v/v) solution. The SAP granules were dried in 70° C. oven for 30 mins.

The FSC, CRC and AUL of the SAP granules were measured. The FSC, CRC and AUL were 46, 32 and 20 g saline/g of SAP, respectively. The absorption speed of the SAP granules was measured by vortex method mentioned in Example 6. The absorption speed of 20-60 mesh SAP granules was 95 seconds while that of 60-80 mesh SAP granules was 41 seconds. The liquid permeability of the SAP granules was measured by column method mentioned in Example 6. The liquid permeability of 20-60 mesh SAP granules was 103 mL/min.

The bio-based content of the SAP was measured by ASTM D6866 international standard test and the measurement was conducted by a third party testing laboratory. The bio-based content of the surface crosslinked SAP granules was 85.15%.

### Example 8—Formation of SAP Granules with SA, Modified Cellulose and PIA-Gelatin as Core Crosslinking Agent and Diethylenetriamine as Surface Crosslinking Agent

SA and CMC, with weight ratio of 1:3, were dissolved together in DI water to form a 3.5% solution. The pH of the solution was adjusted to 5 using NaOH solution. EDC was added and dissolved into the solution in a 5% weight ratio of SA and CMC. The mixture was allowed to have core activation reaction at room temperature for 1 hour. After that gelatin was dissolved in DI water and was added into the mixture in a 25% weight ratio of SA and CMC. NaOH solution was added to the mixture to deprotonate the amine groups of gelatin. 70% neutralized PIA in a 2% weight ratio of SA and CMC was added to the mixture. The mixture was placed at room temperature for another 1 hour for core crosslinking reaction. After that, the mixture was then placed in a 70° C. oven for drying for 4-6 hours. The dried solid was milled into granules using a centrifuge mill. The core SAP granules were classified based on their particle size by sieving method.

The core SAP granules between 20-60 mesh were immersed and stirred in acetone/water (9:1 v/v) solution for 10 mins. After that, EDC was added to the acetone/water solution in 5% weight ratio of the core SAP granules with continuous stirring at room temperature for 15 mins for surface activation reaction. Then diethylenetriamine was added into the mixture in a 4% mole ratio of EDC followed by continuous stirring at room temperature for 1 hour for surface crosslinking reaction. The SAP granules were separated from the solution and were washed with ethanol/water (7:3 v/v) solution. The SAP granules was dried in 70° C. oven for 30 mins.

The FSC, CRC and AUL of the SAP granules were measured. The FSC, CRC and AUL were 63, 49 and 11.4 g saline/g of SAP, respectively. The absorption speed of the SAP granules was measured by vortex method mentioned in Example 6. The absorption speed of 20-60 mesh SAP granules was 68 seconds while that of 60-80 mesh SAP granules was 27 seconds. The liquid permeability of the SAP granules was measured by column method mentioned in Example 6. The liquid permeability of 20-60 mesh SAP granules was 18.2 mL/min while that of 60-80 mesh SAP granules was 3.8 mL/min.

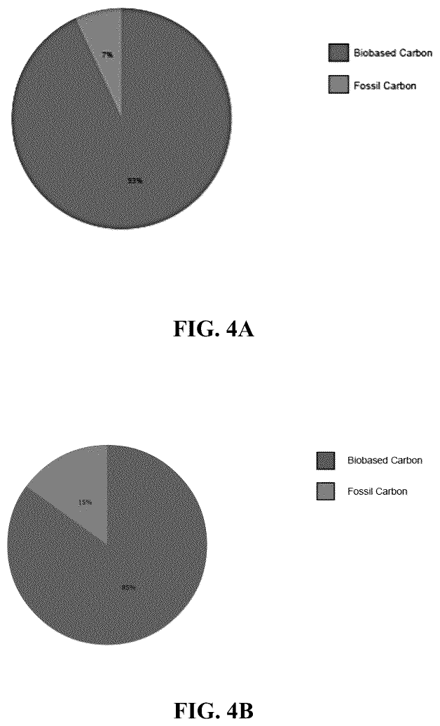

FIG. 4A-4B depicts bio-based content test of the bio-based and biodegradable SAP of the present invention. The bio-based content of the SAP was measured according to ASTM D6866 international standard test and the measurement was conducted by a third party testing laboratory. The bio-based content of the core SAP granules prepared by the formulation of Example 3 was 93%, and that of the surface crosslinked SAP granules prepared by this formulation is 84.47%. These results confirm that the SAP is predominantly derived from renewable plant-based sources.

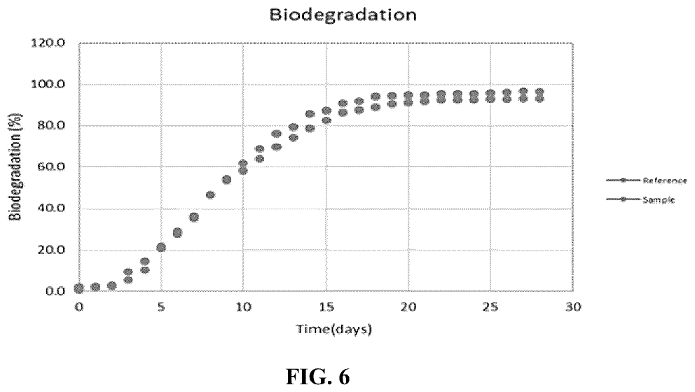

The bio-degradation of the SAP was measured according to OECD 301B CO2 Evolution Ready Biodegradability Test and the measurement was conducted by a third party testing laboratory. FIG. 6 illustrates the main experimental results of the OECD 301B CO2 Evolution Ready Biodegradability Test of the bio-based and biodegradable SAP of the present invention. The SAP showed 93% degradation during 28 days according to the OECD 301B regulation.

The material safety of the SAP granules was analyzed according to REACH SVHC regulation. The SAP granules could pass the regulation. The material safety of the SAP granules was evaluated in accordance with the REACH SVHC (Substances of Very High Concern) analysis by a certified third-party laboratory. The analysis confirmed that the SAP granules contained none of the 241 substances currently listed under the REACH Candidate List, indicating full compliance with EU REACH regulations and suitability for applications in hygiene and skin-contact products.

The skin compatibility of the SAP granules was analyzed according to the regulations of ISO 10993-10:2021 tests for skin sensitization and ISO 10993-23:2021 test for irritation. The analyses were conducted by a third party testing laboratory. In accordance with the Skin Sensitization Test (Guinea Pig Maximization Test, GPMT method) of ISO 10993-10:2021, the SAP granules were extracted using both polar (0.9% sodium chloride) and non-polar (corn oil) extraction vehicles and were extracted at 37° C. for 72 h. The extractions were applied to the testing animals by intradermal injection followed by topical application. By observation of degree of erythema, the results show that the positive rates of all test groups using both polar and non-polar extraction vehicles were 0%. The positive rate of all negative control groups was 0% and the positive rate of all positive control groups was 100%. The results showed no sensitization reactions among test animals, confirming the SAP is non-sensitizing and skin-safe for human use. In addition, the SAP granules were tested under ISO 10993-23:2021 test for dermal irritation using an intradermal injection model. In accordance with the ISO 10993-23:2021, the SAP granules were extracted using both polar (0.9% sodium chloride) and non-polar (corn oil) extraction vehicles and were extracted at 37° C. for 72 h. The extractions were applied to the testing animals by intradermal injection. Degree of erythema and oedema after several time intervals of injection were observed. Results show that the final irritation scores of both polar and non-polar were 0. The final irritation score (polar) of positive control was 5.0. The final irritation score (non-polar) of positive control was 3.5. The test revealed no observable signs of erythema or oedema, and the irritation index fell within the acceptable range defined by the standard, further supporting its biocompatibility.

### Example 9—Formation of SAP Granules with SA, Modified Cellulose, PIA and Surfactant-Gelatin as Core Crosslinking Agent and Jeffamine® T5000 as Surface Crosslinking Agent

SA and CMC, with weight ratio of 1:3, were dissolved together in DI water to form a 3.5% solution. The pH of the solution was adjusted to 5 using NaOH solution. EDC was added and dissolved into the solution in a 5% weight ratio of SA and CMC. The mixture was allowed to have core activation reaction at room temperature for 1 hour. After that gelatin was dissolved in DI water and was added into the mixture in a 25% weight ratio of SA and CMC. NaOH solution was added to the mixture to deprotonate the amine groups of gelatin. 50% neutralized PIA in a 2% weight ratio of SA and CMC and decyl glucoside in a 6.25% weight percent of SA and CMC were added to the mixture. The mixture was placed at room temperature for another 1 hour for core crosslinking reaction. After that, the mixture was then placed in a 70° C. oven for drying for 4-6 hours. The dried solid was milled into granules using a centrifuge mill. The core SAP granules were classified based on their particle size by sieving method.

The core SAP granules between 20-60 mesh were immersed and stirred in acetone/water (9:1 v/v) solution for 10 mins. After that, EDC was added to the acetone/water solution in 5% weigh ratio of the core SAP granules with continuous stirring at room temperature for 15 mins for surface activation reaction. Then Jeffamine® T5000 was added into the mixture in a 4% mole ratio of EDC followed by continuous stirring at room temperature for 60 mins for surface crosslinking reaction. The SAP granules were separated from the solution and were washed with ethanol/water (7:3 v/v) solution. The SAP granules was dried in 70° C. oven for 30 mins.

The FSC, CRC and AUL of the SAP granules were measured. The FSC, CRC and AUL were 48, 33 and 20.8 g saline/g of SAP, respectively. The absorption speed of the SAP granules was measured by vortex method mentioned in Example 6. The absorption speed of 20-60 mesh SAP granules was 45 seconds while that of 60-80 mesh SAP granules was 25 seconds. The liquid permeability of the SAP granules was measured by column method mentioned in Example 6. The liquid permeability of 20-60 mesh SAP granules was 64 mL/min while that of 60-80 mesh SAP granules was 34 mL/min.

The bio-based content of the SAP was measured by ASTM D6866 international standard test and the measurement was conducted by a third party testing laboratory. The bio-based content of the surface crosslinked SAP granules was 85.83%.

The material safety of the SAP granules was analyzed according to REACH SVHC regulation. The SAP granules could pass the regulation.

The skin compatibility of the SAP granules was analyzed according to the regulations of ISO 10993-10:2021 tests for skin sensitization and ISO 10993-23:2021 test for irritation. The analyses were conducted by a third party testing laboratory. The Skin Sensitization Test (GPMT method) of ISO 10993-10 shows that the SAP granules has no skin sensitization reactivity while the ISO 10993-23 Intradermal Reaction Test shows that the SAP granules could meet the standard requirement.

### Example 10—Formation of SAP Granules with SA, Modified Cellulose and PIA-Gelatin as Core Crosslinking Agent and Jeffamine® T5000 as Surface Crosslinking Agent

SA and CMC, with weight ratio of 1:3, were dissolved together in DI water to form a 3.5% solution. The pH of the solution was adjusted to 5 using NaOH solution. EDC was added and dissolved into the solution in a 5% weight ratio of SA and CMC. The mixture was allowed to have core activation reaction at room temperature for 1 hour. After that gelatin was dissolved in DI water and was added into the mixture in a 25% weight ratio of SA and CMC. NaOH solution was added to the mixture to deprotonate the amine groups of gelatin. 50% neutralized PIA in a 2% weight ratio of SA and CMC was added to the mixture. The mixture was placed at room temperature for another 1 hour for core crosslinking reaction. After that, the mixture was then placed in a 70° C. oven for drying for 4-6 hours. The dried solid was milled into granules using a centrifuge mill. The core SAP granules were classified based on their particle size by sieving method.

The core SAP granules between 20-60 mesh were immersed and stirred in acetone/water (9:1 v/v) solution for 10 mins. After that, EDC was added to the acetone/water solution in 3% weigh ratio of the core SAP granules with continuous stirring at room temperature for 15 mins for surface activation reaction. Then Jeffamine® T5000 was added into the mixture in a 4% mole ratio of EDC followed by continuous stirring at room temperature for 60 mins for surface crosslinking reaction. The SAP granules were separated from the solution and were washed with ethanol/water (7:3 v/v) solution. The SAP granules was dried in 70° C. oven for 30 mins.

The FSC, CRC and AUL of the SAP granules were measured. The FSC, CRC and AUL were 45, 31 and 20.6 g saline/g of SAP, respectively. The absorption speed of the SAP granules was measured by vortex method mentioned in Example 6. The absorption speed of 20-60 mesh SAP granules was 110 seconds while that of 60-80 mesh SAP granules was 57 seconds. The liquid permeability of the SAP granules was measured by column method mentioned in Example 6. The liquid permeability of 20-60 mesh SAP granules was 35 mL/min while that of 60-80 mesh SAP granules was 63 mL/min.

The bio-degradation of the SAP was measured according to OECD 301B CO2 Evolution Ready Biodegradability Test and the measurement was conducted by a third party testing laboratory. The SAP showed 68% degradation during 16 days according to the OECD 301B regulation.

### Comparative Example 1

Benchmark SAP 1 composed of polyacrylate was measured with FSC, CRC and AUL. The FSC, CRC and AUL were 62, 37 and 21.8 g saline/g of SAP, respectively. The absorption speed of the SAP granules was measured by vortex method mentioned in Example 6. The absorption speed of 20-60 mesh SAP granules was 48 seconds while that of 60-80 mesh SAP granules was 25 seconds. The liquid permeability of the SAP granules was measured by column method mentioned in Example 6. The liquid permeability of 20-60 mesh SAP granules was 120 mL/min while that of 60-80 mesh SAP granules was 29 mL/min.

### Comparative Example 2

Benchmark SAP 2 composed of polyacrylate was measured with FSC, CRC and AUL. The FSC, CRC and AUL were 52, 28 and 25.6 g saline/g of SAP, respectively. The absorption speed of the SAP granules was measured by vortex method mentioned in Example 6. The absorption speed of 20-60 mesh SAP granules was 43 seconds. The liquid permeability of the SAP granules was measured by column method mentioned in Example 6. The liquid permeability of 20-60 mesh SAP granules was 164 mL/min.

### INDUSTRIAL APPLICABILITY

The bio-based and bio-degradable superabsorbent materials or granules provided in the present disclosure are able to absorb large amount of liquid relative to their own mass but do not dissolve in water. It has many different potential applications, such as agricultural water retention agent, the ingredient material of baby diapers, adult incontinent diapers or female sanitary napkins. The bio-based and bio-degradable SAP has a very high bio-based content as it is synthesized from bio-based polymers or monomers with minimal use of petroleum-based regents. The bio-based and bio-degradable SAP can also fulfil the bio-degradation regulation without phytotoxicity. It is a more sustainable replacement of petroleum-based SAPs especially for the application of single-use products.

What is claimed is:

1. A bio-based and biodegradable superabsorbent polymer, comprising sodium alginate, modified cellulose, at least one activation agent, at least one crosslinking agent and at least one pH-adjusting agent, wherein the bio-based and biodegradable superabsorbent polymer has a bio-based content from 0.1% to 93% and fulfills as least one bio-degradation regulation selected from EN 13432, ASTM D6400, OECD 301B, ISO 17088, EN 14995 or AS 5810.

2. The bio-based and biodegradable superabsorbent polymer of claim 1, wherein the bio-based and biodegradable superabsorbent polymer has a bio-based content from 20% to 93%.

3. The bio-based and biodegradable superabsorbent polymer of claim 1, wherein the modified cellulose comprises carboxymethyl cellulose (CMC), carboxymethyl guar (CMG), hydroxyethyl cellulose (HEC), hydroxypropyl cellulose (HPC), carboxymethyl starch (CMS), or a combination thereof, and is present in an amount of 25 wt % to 85 wt %.

4. The bio-based and biodegradable superabsorbent polymer of claim 1, wherein the at least one activation agent comprises a core activation agent or a surface activation agent.

5. The bio-based and biodegradable superabsorbent polymer of claim 4, wherein the at least one activation agent comprises 1-ethyl-3-(3-dimethylaminopropyl)carbodiimide hydrochloride, dicyclohexylcarbodiimide, N-hydroxysulfosuccinimide, or a combination thereof, and is operable under pH 4 to 7 and ambient temperature conditions.

6. The bio-based and biodegradable superabsorbent polymer of claim 1, wherein the at least one crosslinking agent comprises a core crosslinking agent or a surface crosslinking agent.

7. The bio-based and biodegradable superabsorbent polymer of claim 6, wherein the core crosslinking agent comprises gelatin, chitosan, diethylenetriamine, propane-1,2,3-triamine, polyethylenimine, or a combination thereof; and the surface crosslinking agent comprises diethylenetriamine, propane-1,2,3-triamine, tris(2-aminoethyl)amine, polyetheramines having at least two terminal primary amine groups and a polypropylene glycol or polyethylene glycol or poly(oxypropylene-co-oxyethylene) backbone with molecular weights ranging from approximately 400 to 5000 g/mol, or a combination thereof.

8. The bio-based and biodegradable superabsorbent polymer of claim 1, wherein the at least one pH-adjusting agent comprises hydrochloric acid solution, acetic acid solution, phosphoric acid solution, nitric acid solution, ammonium solution, potassium hydroxide, potassium hydroxide solution, sodium hydroxide, sodium hydroxide solution, or a combination thereof.

9. The bio-based and biodegradable superabsorbent polymer of claim 1, wherein the bio-based and biodegradable superabsorbent polymer further comprises at least one surfactant, itaconic acid, acrylamide, at least one radical initiator and at least one di-acrylate.

10. The bio-based and biodegradable superabsorbent polymer of claim 9, wherein the at least one surfactant comprises octylphenol ethoxylates having an average ethylene oxide chain length of approximately 7 to 70 ethylene oxide units, alkyl polyglucosides comprising coco glucoside, decyl glucoside, octyl glucoside, lauryl glucoside, sucrose cocoate, C₆ alkyl glycoside, C₈₋₁₀ alkyl glycoside, or a combination thereof, and is present in an amount of 0.1 wt % to 10 wt %.

11. The bio-based and biodegradable superabsorbent polymer of claim 9, wherein the at least one radical initiator comprises tert-butyl hydroperoxide, benzoyl peroxide, ammonium persulfate, di-tert-butyl peroxide, potassium persulfate, or a combination thereof.

12. The bio-based and biodegradable superabsorbent polymer of claim 9, wherein the at least one di-acrylate comprises poly(propylene glycol) diacrylate, poly(ethylene glycol) diacrylate, N,N′-methylenebis(acrylamide), tri(ethylene glycol) diacrylate, 1,6-hexanediol diacrylate, or a combination thereof.

13. The bio-based and biodegradable superabsorbent polymer of claim 1, wherein the bio-based and biodegradable superabsorbent polymer is formed into granules with a particle size of 20-100 mesh, and exhibits a free swelling capacity (FSC) of at least 36 g/g, a centrifuge retention capacity (CRC) of at least 24 g/g, and an absorption under load (AUL) of at least 8 g/g.

14. A method for preparing a bio-based and biodegradable superabsorbent polymer, comprising:
dissolving sodium alginate and modified cellulose with weight ratio of 1:0.3-6 in DI water to form the polysaccharide solution;
core crosslinking of the polysaccharide solution to form a core-crosslinked gel;
drying the core-crosslinked gel at 50° C. to 110° C. to form a core superabsorbent polymer;
milling and sieving the core superabsorbent polymer to form one or more core superabsorbent polymer granules with a mesh size smaller than 100 mesh;
surface crosslinking of the one or more core superabsorbent polymer granules to form the one or more superabsorbent polymer granules; and
drying the one or more superabsorbent polymer granules to form the bio-based and biodegradable superabsorbent polymer,
wherein the bio-based and biodegradable superabsorbent polymer exhibits a bio-based content greater than 80%, a free swell capacity of at least 36 g/g saline solution, and undergoes at least 90% degradation within 180 days in accordance with the EN 13432 standard or at least 60% degradation within 28 days in accordance with the OECD 301B standard.

15. The method according to claim 14, wherein step of dissolving sodium alginate and modified cellulose in DI water to form the polysaccharide solution further comprises adding at least one surfactant, itaconic acid, acrylamide, at least one radical initiator and at least one di-acrylate into the polysaccharide solution.

16. The method according to claim 15, wherein the itaconic acid is further subjected to radical polymerization to form a polymer of itaconic acid, and the polymerization of the itaconic acid is conducted at a temperature of 50° C. to 90° C. under nitrogen, optionally with acrylamide in a molar ratio of 3-20:1 and one or more di-acrylates.

17. The method according to claim 14, wherein step of dissolving sodium alginate and modified cellulose in DI water to form the polysaccharide solution comprising:
adding and stirring the sodium alginate and the modified cellulose in the DI water until fully dissolved; and subsequently adding at least one pH-adjusting agent while stirring to adjust the pH of the polysaccharide solution to a range of 4 to 7.

18. The method according to claim 14, wherein the step of core crosslinking of the polysaccharide solution to form a core-crosslinked gel comprising:
adding at least one core activation agent to the polysaccharide solution;
placing the polysaccharide solution at room temperature to allow core activation;
dissolving at least one core crosslinking agent in DI water to form a core crosslinking agent solution;
mixing the core crosslinking agent solution with the polysaccharide solution to form a mixture;
adding at least one pH-adjusting agent while stirring to adjust the pH of the mixture to a range of 7 to 12 and placing the mixture at room temperature to allow core crosslinking to form the core-crosslinked gel.

19. The method according to claim 14, wherein step of surface crosslinking of the one or more core superabsorbent polymer granules comprising:
stirring the core superabsorbent polymer granules in at least one solvent at room temperature;
adding at least one surface activation agent to the core superabsorbent polymer granules while stirring at room temperature to allow surface activation;
adding at least one surface crosslinking agent to the core superabsorbent polymer granules while stirring at room temperature to allow surface crosslinking to form the superabsorbent polymer granules; and
washing the superabsorbent polymer granules with the at least one solvent.

20. The method according to claim 14, wherein step of drying the one or more superabsorbent polymer granules to form the bio-based and biodegradable superabsorbent polymer comprises drying the one or more superabsorbent polymer granules at 50° C. to 110° C.
